(19) United States
(12) Patent Application Publication
(10) Pub. No.: US 2011/0054498 A1
(43) Pub. Date: Mar. 3, 2011

(54) ENDOSCOPIC COMPRESSION CLIP AND SYSTEM AND METHOD FOR USE THEREOF

(75) Inventors: Leonid Monassevitch, Hadera (IL); Boaz Shenhav, Tel Aviv (IL); Shahar Millis, Pardes Hanna (IL); Kobby Greenberg, Even Yehuda (IL)

(73) Assignee: NITI SURGICAL SOLUTIONS LTD., Netanya (IL)

(21) Appl. No.: 12/990,823

(22) PCT Filed: May 5, 2009
(86) PCT No.: PCT/IL09/00466

§ 371 Date: Nov. 3, 2010

Related U.S. Application Data
(60) Provisional application No. 61/071,539, filed on May 5, 2008.

(51) Int. Cl. A61B 17/122; A61B 17/128

(52) U.S. Cl. 606/142; 606/157

(57) ABSTRACT

A compression clip assembly for compressing tissue and operable by means of a user-operated applier. The assembly comprises a compression clip and a lock element. The clip includes a pair of normally spaced apart elongate members each having an outward-facing surface, and having respective inward-facing opposing surfaces for compressing tissue; a hinge formed at least partly of a superelastic material and in operative mechanical connection with the elongate members; and a lock region formed on the outer surface of each of the elongate members adjacent to the hinge, each lock region being delimited by a first stop element proximal to the hinge and a second stop element distal from the hinge. The lock element is constructed for lockably engaging the lock regions so as to lock the clip in its closed position. The invention also encompasses a system and method for using the compression clip assembly.

### FIELD OF THE INVENTION

The present invention relates to endoscopic compression clips and a method and system for their use.

### BACKGROUND OF THE INVENTION

Polyps are defined as growths or masses protruding from a mucous membrane of the body. They may occur in the mucous membrane of many different types of organs, such as the nose, mouth, stomach, intestines, rectum, urinary bladder, and uterus. Most polyps are benign and eventually stop growing, but some may ultimately become cancerous tumors. Colorectal or gastric cancers, often beginning as benign or precancerous polyps, can essentially be avoided if detected and treated in their early stages by performing a polypectomy.

Polypectomy is the medical term for removing polyps, particularly small polyps of the colon and stomach. These can be removed by using a biopsy forceps, which removes small pieces of tissue. Larger polyps are usually removed by putting a noose, or snare, around the polyp base or stalk and burning through the tissue with an electric instrument (cauterization). Other devices employ physical or electrical scraping of the lining of an organ, such as the colon, rectum or stomach, to remove the polyp. The severed polyps are usually retrieved for examination by a pathologist.

Complications, however, sometimes occur during polypectomies. Particularly problematic is bleeding induced by the device used to resect the polyp. The bleeding may be immediate or delayed. When bleeding is immediate, the vision of the physician is obscured and this may interfere with the completion of the surgical procedure, often an endoscopic procedure. When bleeding is delayed, additional surgical intervention, even possibly full invasive surgery may be required.

Mechanical surgical clips, particularly compression clips, for use in endoscopic surgery, including endoscopic polypectomies, are known. These clips inter alia are intended to achieve hemostasis as they apply constrictive forces to blood vessels so as to limit or interrupt blood flow. In effect, the bleeding vessel is ligated, or the tissue around the bleed site is compressed, ligating all of the surrounding vessels. However, these clips have drawbacks.

The typical clip is a two legged clip that is passed through an endoscope's working channel via a flexible delivery catheter. Because the clip needs to pass through the endoscope, the clip's size is limited. Size limitations prevent the clip from being able to effectively clamp off all of the blood vessel or vessels in the tissue or polyp's stalk to be resected. Additionally, the clip may be unable to provide sufficient clamping force because of its structural design.

An additional problem with these clips is that when delivering these clips to the wound site, good visualization of a bleeding vessel cannot be obtained. The endoscopist may be required to blindly attach the clip, resulting in an imprecisely performed procedure that requires guess work on the part of the endoscopist. Attaching a clip therefore may be unsuccessful during an initial attempt.

Finally, many clips slip off the tissue to which they have been attached and are compressing during, or subsequent to, a surgical procedure but prior to complete healing of the wound. All this leads to frequent follow-up endoscopic surveillance, adding to patient discomfort and extra costs to the health care system.

Other medical conditions also make use of compressive hemostatic clips. One such condition is peptic ulcer disease (PUD). This condition is associated with bleeding that can be fatal if not treated immediately. Internal hemorrhaging may be intense and a bleeding peptic ulcer can be a critical clinical event.

Suspected bleeding PUD patients can be diagnosed and treated endoscopically in an emergency room, an ICU or the GI suite. Many of the treatments used on PUDs, such as thermal cauterization, are similar to those applied when endoscopically removing polyps. The main goal in this procedure is to achieve rapid and effective hemostasis. As with polyp removal, delayed bleeding is a problem.

Other lesions, such as fistulas and organ perforations, the latter either naturally occurring or surgically produced, are also susceptible to bleeding.

Therefore, there remains a need for an endoscopic compression clip and a system and a method which would facilitate its use, particularly in endoscopically carried out polypectomies. The clip should be reopenable to ensure that if initial positioning is unsuccessful, the clip can be easily repositioned. In general, there is a need for such a clip, system and method that could be applied to stanch bleeding from all types of bodily lesions, naturally occurring or surgically generated.

### TERMINOLOGY

“Proximal” relates to the side of an endoscope, a clip, a device or an element closest to the user, while “distal” refers to the side of the endoscope, the clip, the device or the element furthest from the user.

“Polyp” as used in the specification and claims herein is not intended to restrict the assembly, system, subsystems, elements and method discussed herein to polyps alone. Other types of suspect lesions may also be treated using the assembly, system, subsystems, elements and method discussed herein.

“Lesion”, in addition to its use herein to refer to many different types of localized pathological changes in a body organ or tissue, may also be used herein in place of the word “polyp” without any intent at differentiating between the two terms except where specifically indicated. Lesion also contemplates fistulas and organ perforations, either naturally occurring perforations or perforations produced during surgical procedures.

“Tissue” includes, but is not limited to, tissue of the gastrointestinal tract, and the vascular system. The assembly, system, subsystems, elements and method discussed herein may be used with tissue of any internal organ.

“Gastrointestinal tract” or its equivalents are used in the specification and claims without the intent of being limiting. Other organ systems, and lesions found therein, are also contemplated as being treatable with the assembly system, subsystems, elements and methods discussed in the present specification.

“Hinge” is a force applier and this latter term may be used herein interchangeably with hinge, hinge spring or clip hinge without any intent at differentiating between any of these terms, except where specifically indicated.

“Endoscope”, as used herein, should be construed as including all types of invasive instruments, flexible or rigid, having scope features. These include, but are not limited to, colonoscopes, gastroscopes, laparoscopes, and rectoscopes. Similarly, the use of “endoscopic” is to be construed as referring to all types of invasive scopes.

“Endoscopist” as used herein refers to any user of the clips and the clip system described herein. Besides a physician, it may refer to any other properly trained medical personnel.

“Applier” as used herein may be used interchangeably with the term “delivery system” without any attempt at differentiating between them. The applier delivers a compression clip assembly constructed according to an embodiment of the present invention, positions it and locks it around tissue of a lesion to be compressed.

### SUMMARY OF THE INVENTION

It is an object of the present invention to provide an endoscopic compression clip (ECC), often denoted herein as “clip”, for compression of a lesion, typically but without intending to limit the invention, a gastrointestinal (GI) lesion such as a polyp.

It is a further object of the present invention to provide a clip that can inter alia be used for hemostasis for mucosal or sub-mucosal defects, arteries, diverticula in the colon, for endoscopic marking, for anchoring or otherwise affixing jejunal feeding tubes, closing perforations, either naturally occurring or surgically produced, and multiple clip compression uses.

An additional object of the present invention is to provide a compression clip that reduces the incidence of procedure-related bleeding, irrespective of whether the bleeding is immediate or delayed.

Yet another object of the present invention is to provide a compression clip which is deployed in a controlled fashion and where the arms, that is the elongate members, of the clip can be reopened and repositioned any number of times prior to locking the clip into its final position.

Another object of the present invention is to provide a compression clip with a wide-angle opening between its elongate members

It is an object of the present invention to provide a system and method for use with an endoscopic compression clip constructed and operative according to embodiments of the present invention.

In one aspect of the present invention, there is provided a compression clip assembly for compressing tissue and operable by means of a user-operated applier. The assembly includes:
A. an endoscopic compression clip having open and closed positions, wherein the clip includes:
(i) a pair of normally spaced apart elongate members each having an outward-facing surface, and having respective inward-facing opposing surfaces for holding and compressing tissue;
(ii) a hinge formed at least partly of a superelastic material and in operative mechanical connection with the elongate members; and
(iii) a lock region formed on the outer surface of each of the elongate members adjacent to the hinge, each of the lock regions being delimited by a first stop element proximate to the hinge and a second stop element distal from the hinge; and
B. a lock element for lockably engaging the lock regions so as to lock the clip in its closed position, the lock element and the lock regions being formed so as to facilitate relative translation of the lock element and the lock region until the lock element is positioned between the first and second stop elements.

In an embodiment of the compression clip assembly, the assembly further includes one or more third elements in each lock region. The one or more third elements are positioned between the first and second stop elements. Locking of the clip is effected when the one or more third elements are forcibly engaged by and pass within the lock element. This results in a required increase in force for further movement of the clip through the lock element so as to lock the clip with the lock element.

In another embodiment of the assembly, the lock element further includes one or more orientation teeth and one or more male yoke members for disengageably mating with the user-operated applier.

In yet another embodiment of the assembly, the lock element locks the clip after being positioned against the second stop elements.

In still another embodiment of the assembly, the hinge is configured as a substantially closed geometric shape enclosing an area large enough to accommodate a means for mechanical connection of the applier.

In a further embodiment of the assembly, the elongate members of the clip are formed of a superelastic material.

In yet another embodiment of the assembly, when the clip is locked, the ratio of the length of the elongate members of the clip extending past the lock element to the length of the lock element itself is from about 1 to about 7.

In another aspect of the invention, there is provided a system for applying a compression clip for compressing tissue. The system includes:
A. a compression clip assembly, the assembly including:
   (a) an endoscopic compression clip having open and closed positions wherein the clip includes:
      (i) a pair of normally spaced apart, elongate members each having an outward-facing surface, and having respective inward facing opposing surfaces for holding and compressing tissue;
      (ii) a hinge at least partly formed of a superelastic material and in operative mechanical connection with the elongate members; and
      (iii) a lock region formed on the outer surface of each of the elongate members adjacent to the hinge, each of the lock regions being delimited by a first stop element proximate to the hinge and a second stop element distal from the hinge; and
   (b) a lock element lockably engaging the lock regions so as to lock the clip in its closed position, the locking element and the lock regions being formed so as to facilitate relative translation of the lock element and the lock region until the lock element is positioned between the first and second stop elements;
B. an applier which includes:
(a) a housing having a periphery with a pair of slots symmetrically positioned therein and disengageably mateable with the lock element;
(b) a force transmitting element positioned within the housing and including two arms formed of a resilient material, each of the arms having a free end and insertion elements formed thereat for insertion into the hinge;
(c) a control means operative to selectably move the force transmitting element in the direction of the first stop elements and in the direction of the second stop elements of the clip; and
C. means for applying a resistive force operative to indicate that applying force to overcome the resistive force will lock the clip.
The control means may be selectably moved by a user in a selected one of the proximal and distal directions causing the force transmitting element to move by a preselected distance. The preselected distance is determined by the encounter of a resistive force when the clip is pulled in the proximal direction, the resistive force provided by the means for applying a resistive force so as to oppose movement of the clip within the lock element. When the force transmitting element is pulled so as to move the clip beyond the preselected distance overcoming the increased resistive force, the clip is positioned so that the lock element locks the clip in its closed position and the pair of elongate members of the clip are positioned adjacent to each other thereby to compress tissue held therebetween. The insertion elements pull away and disengage from the hinge and the force transmitting element arms exit the slots.

In another embodiment of the system, the lock element further includes one or more male yoke members and one or more orientation teeth and the housing further includes one or more yoke elements and one or more housing orientation spaces for disengageably mating with the one or more male yoke members and the one or more orientation teeth, respectively.

In yet another embodiment of the system, the resilient material of the force transmitting element arms is a superelastic material. When the force transmitting element is pulled so as to move beyond the preselected distance, the force transmitting element arms, confined in the housing, are operative to disengage from the hinge and to spring open and exit the housing slots after disengaging from the clip.

In still another embodiment of the system, when the force transmitting element is pulled so as to move beyond the preselected distance, the force transmitting element arms disengage from the clip and then are positioned to push against the one or more male yoke members of the lock element, thereby assisting in disengaging the locked clip assembly from the housing of the applier.

In yet another embodiment of the system, the clip includes one or more third elements. The one or more third elements are positioned between the first and second stop elements. The one or more third elements when encountered serve as the means for applying a resistive force, thereby indicating to the user imminent locking of the clip consequent to further application of force to the control means.

In yet another embodiment of the system, the arms of the force transmitting element include a pair of force transmitting element projections and each of the housing slots has a narrow proximal part and a wider distal part. At the junction of the wider and narrower parts, the parts form a step, the step serving as the means for applying a resistive force, thereby indicating to the user imminent locking of the clip consequent to further application of force to the control means.

In another embodiment of the system, the housing includes one or more yoke elements and the housing is constructed of a material that allows spreading of the one or more housing yoke elements when the force transmitting element projections enter the narrower proximal part of the slots so that the locked clip is more easily disengaged.

In still another embodiment of the system, the resilient material of the force transmitting element is a superelastic material.

In a further embodiment of the system, the open position of the clip forms an angle of at least about 45 degrees.

In still another embodiment of the system, the applier further includes an overtube for compressing the elongate members of the compression clip holding them in their closed position while the clip is brought to tissue to be compressed.

In another aspect of the invention there is provided a method for compressing tissue. The method includes the steps of:
bringing a compression clip assembly, including a compression clip and a lock element, using an applier to tissue to be compressed;
opening and closing the compression clip, as often as necessary, around the tissue to be compressed until a proper positioning of the clip has been achieved;
locking the clip so that its elongate members are held adjacent to each other compressing the tissue held therebetween; and
freeing the locked clip from the applier by pulling on a compressed resilient force transmitting element of the applier so that it is brought to, and at least partly passes out of, slots in the wall of a housing of the applier removing the compressive force acting on the resilient force transmitting element allowing for disengagement of the clip from the applier.

In another embodiment of the method, the method further includes a step of drawing an overtube over the compression clip prior to the step of bringing and a step of pulling back the overtube and uncovering the clip, allowing the clip to return to its biased open position after the step of bringing.

In yet another embodiment of the method, the step of locking further includes a step of bringing the compression clip through the lock element so that the lock element passes over one or more projections on the clip after which the clip locks, the act of passing over the one or more projections after which the clip locks requires additional force by a user signaling to the user that passing the one or more projections will irreversibly lock the clip.

In still another embodiment of the method, the step of freeing further includes a step of moving the resilient force transmitting element so as to press against elements on the lock element mateable with elements on the housing of the applier to further assist in disengagement of the locked clip from the applier.

In yet another embodiment of the method, the step of locking further includes a step of bringing projections located on the resilient force transmitting element over a juncture formed by a narrower portion and a wider portion of the housing slots, the juncture requiring additional force by a user signaling to the user that passing the juncture will irreversibly lock the clip.

In still another embodiment of the method, the step of freeing includes a step of pulling the resilient force transmitting element so that the projections thereon enter the narrower part of the housing slots thereby locking the clip and facilitating disengagement of the locked clip from the applier by spreading apart elements of the housing mateably engaged with elements on the lock element.

### BRIEF DESCRIPTION OF THE DRAWINGS

The present invention will be more fully understood and its features and advantages will become apparent to those skilled in the art by reference to the ensuing description, taken in conjunction with the accompanying drawings, in which:

FIGS. 1A-1C are side views of the actuator assembly, delivery system, and deployment assembly of an applier operative to deliver and activate a compression clip assembly constructed according to embodiments of the present invention, the clip being shown in its insertion, clip opening, and clip closing stages;

FIG. 1D shows the mechanism of actuation of the actuator assembly shown in FIGS. 1A-1C;

FIGS. 2A-2E show various views of a compression clip assembly, constructed according to an embodiment of the present invention;

FIGS. 3A and 3B show two isometric views of a force transmitting element constructed in accordance with an embodiment of the present invention, one view of which shows the force transmitting element positioned in the housing of the applier's deployment assembly;

FIG. 4 is an isometric view of the endoscopic compression clip (ECC), clip lock element, and force transmitting element constructed according to an embodiment of the present invention;

FIGS. 5A-5D show several views of the clip, clip lock element and distal end of the applier's deployment assembly at various stages of the ECC and clip lock element's operation according to the method of the present invention;

FIGS. 6A-6D show different views of the pre-disengagement and disengagement steps of the compression clip assembly according to the present invention;

FIGS. 7A-7D are additional views, generally cut-away views, of the clip, clip lock element and deployment assembly at various stages of operation according to the present invention;

FIGS. 8A and 8B are two views of a clipped polyp using a locked endoscopic compression clip assembly constructed in accordance with an embodiment, and applied by the system, of the present invention;

FIGS. 9A-9D show several isometric views of a compression clip assembly constructed according to a second embodiment of the present invention, the clip being presented at various stages of its operation;

FIGS. 10A-10C show several views of a clip constructed according to a third embodiment of the present invention, the compression clip being presented at various stages of its operation; and

FIGS. 11A-11G show several views of a compression clip assembly constructed in accordance with an embodiment of present invention and a second deployment assembly for deploying the compression clip assembly.

Similar elements in the Figures are numbered with similar reference numerals.

### DETAILED DESCRIPTION OF THE PREFERRED EMBODIMENTS

The present invention provides an endoscopic compression clip (ECC) for use in endoscopic procedures, inter alia for use in inducing hemostasis. The clip allows for being opened and closed by the endoscopist an unlimited number of times until satisfactory positioning of the clip is achieved. The clip may then be locked by a clip lock element disengageably connected to the housing of a deployment assembly. The deployment assembly is part of a clip delivery system herein denoted as an applier. The clip lock element is disengaged together with the clip from the deployment assembly of the applier and holds the clip in its locked closed position while the clip is compressing tissue. The clip and clip lock element together form what herein is denoted as the compression clip assembly.

The deployment assembly of the applier includes a force transmitting element. In what is described herein the force transmitting element will often be denoted and described as a fork element, typically, but without intending to limit the invention, having a forceps shape. It should readily be understood by persons skilled in the art that the fork element is exemplary only; other constructions of a force transmitting element may also be used. The force transmitting element is formed of a resilient material which allows it to remain in a closed configuration when under a compressive force supplied by the housing of the applier's deployment assembly.

The clip lock element is formed to contain one or more male yoke members and one or more orientation teeth which are mateably and disengageably joinable to one or more yoke elements of the housing of the applier's deployment assembly and the one or more housing orientation spaces thereof, respectively. Disengagement is effected by the force of pulling the clip in the proximal direction into the clip lock element until the lock element reaches distal stop projections on the clip. If disengagement does not occur immediately upon locking the clip, in some embodiments of the invention the arms of the fork element can be used to assist in disengaging the locked clip assembly as will be described below.

Disengagement of the clip from the applier's deployment assembly occurs only after the endoscopist is satisfied with the positioning of the clip around the tissue to be compressed and only after the lock element engages and lies entirely within the clip's locking region. The locking region is located on the outer surface of each elongate member of the clip adjacent to its hinge. In some embodiments, locking of the clip occurs only after the lock element passes over one or more projections positioned in the locking region near the hinge of the clip. These projections provide a resistive force that indicates that an increase in force is required for the lock element to be pulled past these one or more projections. They alert the endoscopist that application of an increased force will result in locking of the clip, allowing him to desist from applying such force preventing the clip from being locked unintentionally.

In other embodiments, locking of the clip occurs after one or more projections located on the arms of the force transmitting element pass a resistive step in release slots located in the housing of the deployment assembly. The resistive step provides a resistive force that indicates to the endoscopist that an increase in force is required for the clip to be pulled further into the lock element thereby locking the clip. This increase in resistive force prevents the endoscopist from locking the clip unintentionally.

It should be noted that in all embodiments of the invention, it is the lock element of the compression clip assembly that remains stationary while it is the clip that is pulled or pushed through the lock element.

Disengagement of the clip and clip lock element is effected when the arms of the force transmitting element, e.g. fork element, are brought adjacent to a region of the housing of the applier's deployment assembly having release openings, also denoted herein as release slots. These release openings act to release the compressive force operative on the resilient arms of the fork element. The release of the compressive force allows for the spreading apart of the fork element's arms. Insertion elements on the fork element's arms, also denoted herein as fork arm projections, then move out of the hinge loop region formed by the clip hinge and exit through the release openings, thereby disengaging the fork element from the clip.

Once the fork arm projections are released from the clip's hinge, the locked clip assembly detaches from the housing of the deployment assembly by separation of the lock element's one or more male yoke members from the housing's one or more yoke elements which hold them. In the case that the detachment is incomplete, the freed arms of the fork element may then be maneuvered to push against the one or more male yoke members of the lock element causing the lock element to separate from the housing of the applier's deployment assembly.

The point of attachment between the fork arms' projections, that is the fork element's insertion elements, and the clip has been described above as a hinge loop. It should be understood that the clip's hinge may be constructed to form any closed shape with a hole in it in addition to a loop through which the fork arm insertion elements may be inserted. The closed shape, however, should have a sufficient area to accommodate and retain the fork arm insertion elements when inserted. The insertion elements are just one means for mechanical connection of the applier to the clip. It should be appreciated by persons skilled in the art that other such means are possible.

The clip, generally the clip hinge, is at least partly made of a superelastic material. This may be a shape memory alloy which exhibits superelasticity, such as a nickel-titanium (Ni—Ti) alloy. In some embodiments, the remainder of the clip may also be made of a superelastic material. The clip is biased to be in its open position with its arms spaced apart. In its open position, the arms of the clip form an angle equal to or greater than 45°. However, it should be understood that this angle is not intended to limit the invention. When nitinol is used in the hinge, its superelastic characteristic allows for greater elastic deformation, that is, deformation without plastic deformation, thereby allowing for the wide angle opening.

In some embodiments, the material used in the clip hinge may be a shape-memory material and not necessarily a superelastic material.

Nitinol or other superelastic material may also be used in the elongate members, herein also denoted as clip arms. When used in the clip arms, thicker tissue can be effectively compressed as the arms are superelastic and possesses a spring effect over a greater range of deflection then other materials. When nitinol is used only in the hinge, the clip arms may be made of plastic or any other stiff material and may be attached to the hinge by any of many methods known to those skilled in the art.

The present invention contemplates a working length ratio of the clip arms, that is, the elongate members, of from about 1 to about 7, more preferably from about 2 to about 6, and even more preferably from about 3 to about 5. The working length ratio (D/L) is defined herein as the ratio of the length of the clip from its distal end to the lock element when the lock element is in its locking position (D) to the length of the lock element (L). The large ratio provided by clips of the present invention allows for compression of thicker tissue (e.g. polyps with large stalks) and for closure of larger perforations.

In what is described herein, the use of the term open position and closed position for the clip refers to the position of the clip's elongate members. When the clip is in its open position the elongate members, the clip arms, are spaced apart. When the clip is in its closed position, the elongate members are not spaced apart and may be substantially adjacent to each other.

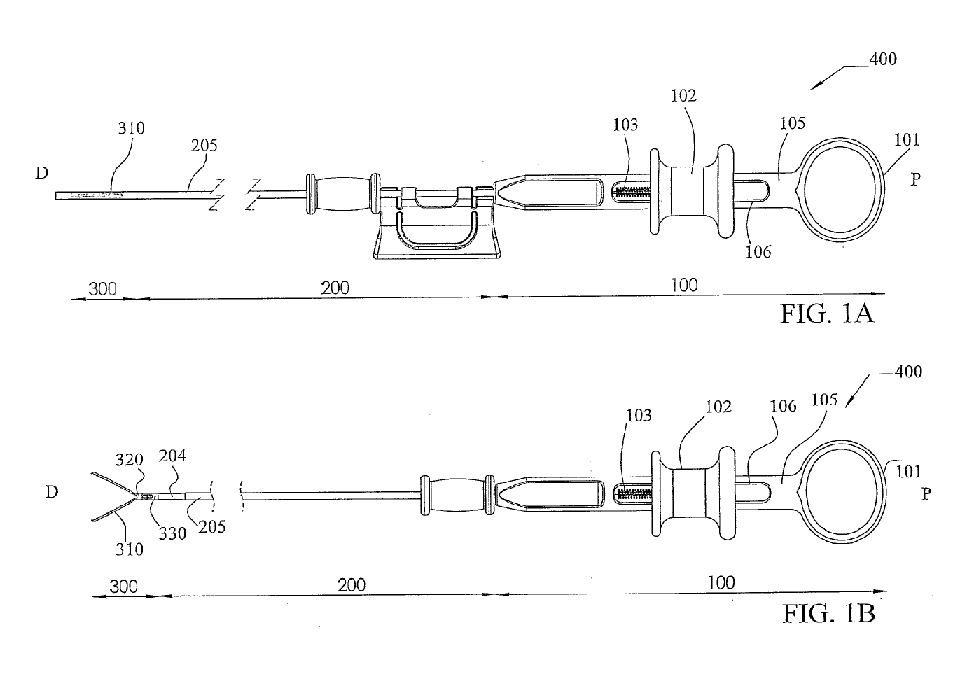

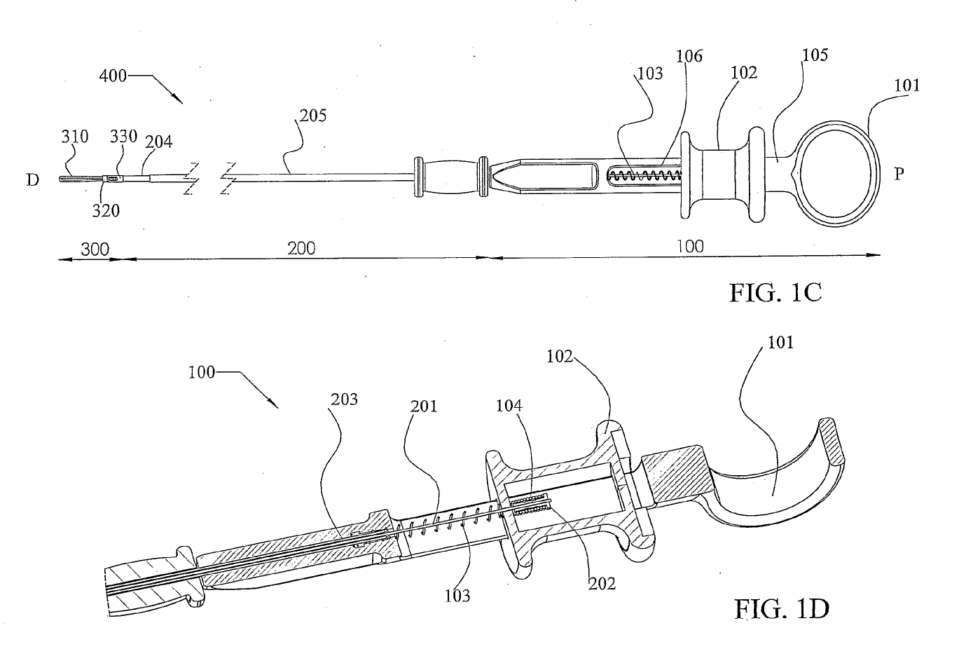

FIGS. 1A-1D, to which reference is now made, show various stages of the deployment of the compression clip of the present invention using a typical, but non-limiting, applier for delivering and applying the clip. FIG. 1A shows a side view of the applier with the clip 310 kept in its closed position by an overtube 205, this being the position in which the clip is inserted into a body lumen using a typical, but non-limiting endoscope. FIG. 1B shows a side view of the applier when the clip 310 has been exposed by pulling the overtube 205 in the proximal direction allowing the clip to move to its biased open position. At this stage, the endoscopist can repeatedly open and close the clip until it is satisfactorily positioned around a lesion. FIG. 1C shows a side view of the applier and clip 310 when the clip has been closed. The actual disengagement of the locked clip from the applier's deployment assembly is not shown. FIG. 1D shows an enlarged cut-away view of the spring mechanism which advances and activates the clip via a control means, typically, but without intending to limit the invention, a control wire 201, as in FIG. 1D.

As shown in FIG. 1A, endoscopic compression clip (ECC) system 400 is comprised of an actuator assembly 100, a delivery section 200, a deployment assembly 300 and a clip 310. Clip 310 and deployment assembly 300 are discussed together in greater detail below.

Actuator assembly 100 may be constructed in a manner similar to conventional actuator assemblies of the type generally employed in endoscopic biopsy devices or in assemblies constructed for other similar applications. These are known to persons skilled in the art. Actuator assembly 100 allows the user to move a control wire 201 or other force transmitter, which is also denoted herein as a control means. Control wire 201 extends through shaft 204 (FIGS. 1B and 1C) to deployment assembly 300 at the distal end D of system 400. Pushing control wire 201 moves clip 310 to, and out of, the distal end D of shaft 204 (FIG. 1B), while pulling wire 201 moves clip 310 in the proximal P direction.

Shaft 204, typically a flexible coil, is designed to provide structural strength and to transmit a torque from its proximal end P to its distal end D. The flexible coil may be a conventional coil used in other biopsy devices and may, for example, comprise a single, coiled wire. The coiled wire may have a round, square or a rectangular cross section, and may be made of a biocompatible material such as, for example, stainless steel. Additional protective and low friction outer layers may be included on control wire 201 and/or shaft 204, according to known methods of construction. Sliding over the distal end D of shaft 204 is overtube 205 (FIGS. 1A and 1B). Shaft 204 may alternatively be constructed as a tube, typically, but without limiting the invention, of plastic that is flexible enough to bend yet transmits force from its proximal to its distal end.

A deployment spring 104, best seen in FIG. 1D, may be provided within the body of actuator assembly 100, positioned within control knob 102 to bias the knob, and thus the control wire 201, toward a desired position. Control knob 102 mounted on actuator body 105 moves by sliding it along a guide slot 106. Deployment spring 104 is in mechanical communication with control wire 201 through wire lock 202. The endoscopist is able to manipulate control wire 201 by grasping thumb holder 101 and moving control knob 102 along guide slot 106.

Spring 104 has a double purpose. First, spring 104 absorbs the relative movement between control wire 201 and shaft 204 produced by the curves of the body lumen into which the endoscope is inserted. This prevents clip 310 from inadvertently being pulled in the proximal direction. Second, deployment spring 104 increases the operating length of knob 102. Deployment spring 104 amplifies the movement of knob 102 since the overall movement of clip 310 and control wire 201 between the clip's open and closed positions is very small. Pulling knob 102 in the proximal direction will not affect wire 201 until deployment spring 104 is fully compressed. Then, any additional movement of knob 102 in the proximal direction will pull wire 201 in that direction.

As shown in FIG. 1D, positioned over wire 201 and in guide slot 106 is tensile spring 103 which keeps knob 102 under slight tension. This tension acting through wire 201 holds clip 310 against clip lock element 320 preventing undesired movement of the clip.

The proximal end of control wire 201 is attached to sliding control knob 102 using any of many methods known to persons skilled in the art. Stainless steel or other high yield biocompatible materials may be used to manufacture control wire 201 so that the structural integrity of the assembly is maintained. A superelastic material, such as nitinol, may also be used to form control wire 201.

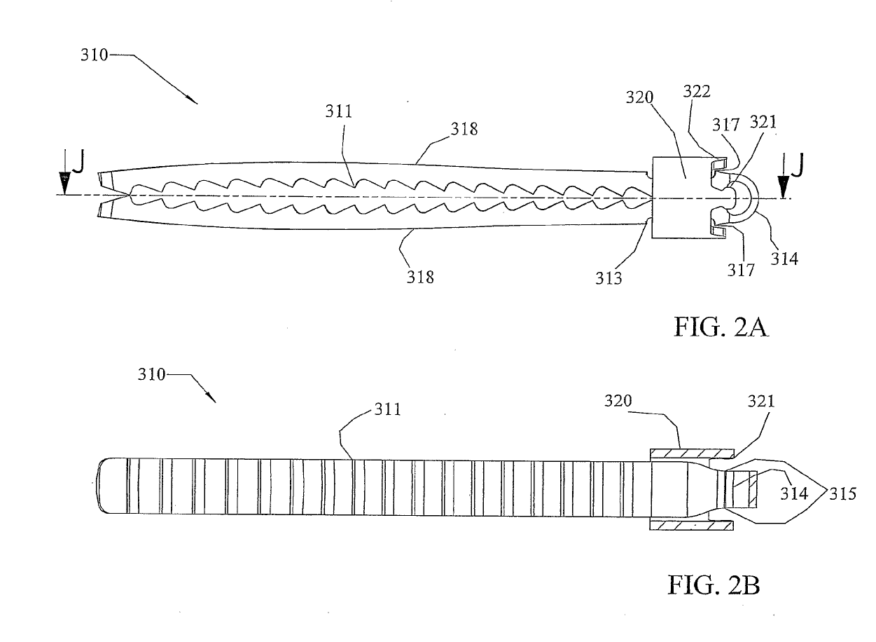

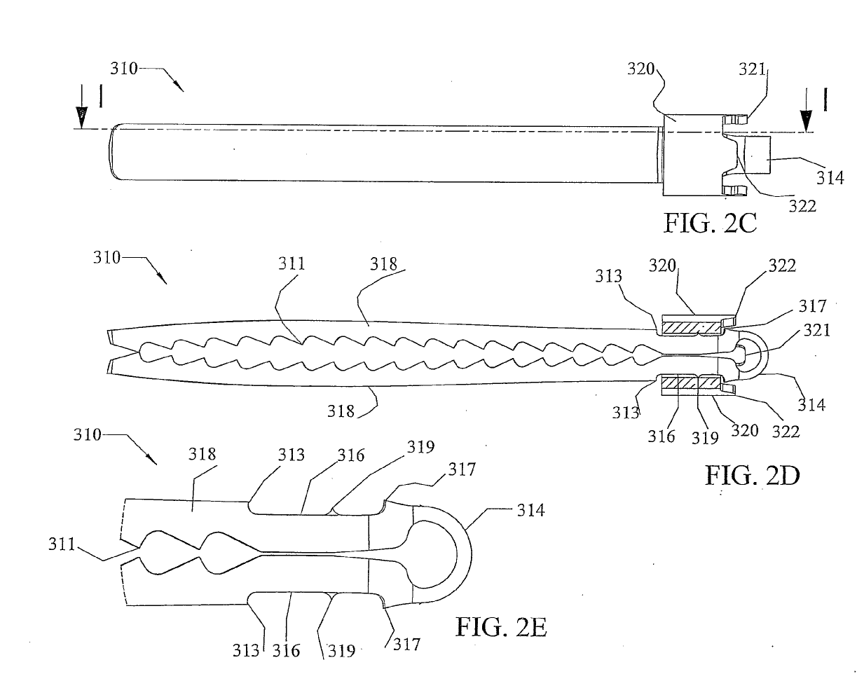

Several views of compression clip 310 and clip lock 320 constructed according to an embodiment of the present invention are shown in FIGS. 2A-2E, to which reference is now made. FIG. 2A is a side view of clip 310 and clip lock element 320; FIG. 2B is a view of one side of the clip viewed from between the arms 318 of clip 310 along a cut through the J-J axis of FIG. 2A; FIG. 2C is a top view of clip 310 and lock element 320; FIG. 2D is a cut-away side view of clip 310 and lock element 320 along a cut through the I-I axis of FIG. 2C; and FIG. 2E is a side view of the hinge 314 region of clip 310 without clip lock element 320.

Clip 310 is at least partially formed of a superelastic material. This may be a shape memory alloy which exhibits this property such as, but without intending to limit the invention, a nickel-titanium (Ni—Ti) alloy. In particular, but again without intending to limit the invention, clip hinge 314 may be formed at least partially of a superelastic material. The two elongate members or arms 318 of clip 310 contain teeth 311 for better grasping the tissue being held, and for preventing the tissue from slipping out of the arms of the clip when the tissue is grasped and the clip is locked. As best seen in FIG. 2E, the proximal end of clip 310 includes clip arms 318, clip hinge 314, lock socket 316, distal stop projections 313, middle stop projections 319 and proximal stop projections 317. Projections 313 and 317 may also be denoted herein as first and second stop elements, respectively. Lock socket 316 may also be denoted herein as lock region 316. Lock region 316 is formed on the outer-facing surface of each of elongate members 318 adjacent to hinge 314. The region is delimited by projections 313 and 317.

Clip lock element 320 is shown in various views in FIGS. 2A-2D. Lock element 320 contains one or more male yoke members 321 and one or more orientation teeth 322. When clip lock element 320 locks clip 310 in its closed grasping position, the lock element moves from a position adjacent to proximal stop projections 317 over middle stop projections 319 (FIG. 2E) and is held in lock socket 316 (FIGS. 2D and 2E) of clip 310 between stop projections 313 and 317 (FIG. 2D). Lock element 320 can not move further in the distal direction because such movement is prevented by distal stop projections 313 (FIG. 2E). Similarly, when clip 310 is deployed and locked, lock element 320 can not fall off clip 310 by moving in the proximal direction—the direction of the clip hinge 314; that is prevented by proximal stop projections 317.

Lock element 320 can not move past projections 313 and 317 because the inner diameter of lock element 320 is smaller than the distance between the projections on opposing clip arms.

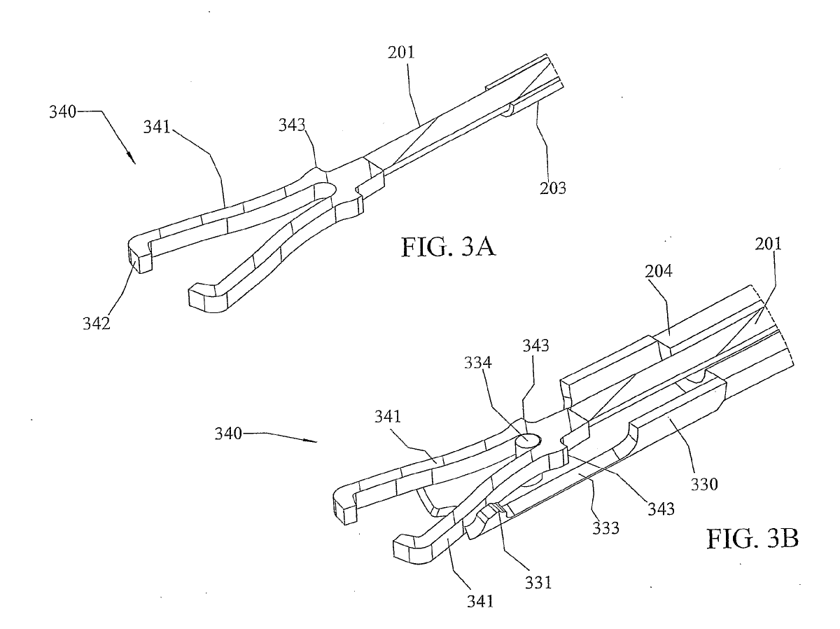

As will be discussed further below, middle stop projections 319 allow for the opening and closing of clip 310 without it being locked. When control wire 201 is pushed in the distal direction as discussed below, force transmitting element, here a fork element, 340 (FIG. 3A, for example), in mechanical communication with clip 310 and control wire 201 (described below), moves clip 310 in the distal direction. When clip 310 is being pulled in the proximal direction and moved relative to clip lock element 320, clip lock element 320 encounters middle stop projections 319. These projections transmit an additional resistive force to the endoscopist indicating that further advance of the clip through lock element 320 in the proximal direction would lock the clip. Therefore, middle stop projections 319 effectively act to prevent the clip from moving to its locked position within clip lock element 320, in lock socket 316 between projections 313 and 317, before the endoscopist is satisfied with the positioning of the clip around the lesion. Projections 319 are also denoted herein as a “means for applying a resistive force operative to indicate that applying force to overcome the resistive force will lock the clip”.

It should readily be appreciated that while force transmitting element 340 is herein described in terms of a fork element, other force transmitting elements may be designed and used. These function essentially as the fork element discussed herein.

There is a gradual narrowing 315 (best seen in FIG. 2B) of the width of clip 310 from the region immediately proximal to projections 317 and in the proximal direction, that is, in the direction of clip hinge 314. This narrowing allows for a better mechanical connection between the fork arm projections 342 of fork element 340 (discussed below) and clip 310.

It should be noted that the present invention contemplates embodiments where the clips may have fewer than three pairs of stop projections but at least a single pair of stop projections. It must have distal stop projections 313 to stop the lock in the distal direction. In some embodiments, the hinge can be designed in a way that allows it to serve as the proximal stop projection.

Connecting clip 310 to the remainder of ECC system 400 discussed in conjunction with FIGS. 1A-1D is force transmitting element, here a fork element, 340 shown in FIGS. 3A and 3B. Fork element 340 is formed of two fork arms 341 each having at its distal end a fork arm projection 342 and is positioned within deployment assembly housing 330. Fork element 340 as seen in FIGS. 3A and 3B has a generally forceps-like shape at the base of which are fork stopper projections 343. These projections stop the fork element from moving further than necessary when the fork is being pulled in the proximal direction by control wire 201. At the proximal end of fork 340, the fork is in mechanical communication with control wire 201 which, as discussed above, extends from actuator assembly 100 (FIGS. 1A-1D) to fork element 340 in a wire cover 203 (FIG. 1D). A wire cover or a coated wire is used to decrease friction between wire 201 and spring shaft 204.

Fork element 340 may be fabricated from any of many different resilient materials including superelastic materials. Accordingly, fork element 340 may at times be denoted herein as a “resilient element 340”. In some embodiments, superelastic materials, such as nitinol, may be used, while in other embodiments, more conventional resilient materials, for example stainless steel, may be used. In general, fork element 340 may be formed from any alloy and mechanically forced into its locked, that is clip holding, configuration once positioned in a housing of the deployment assembly.

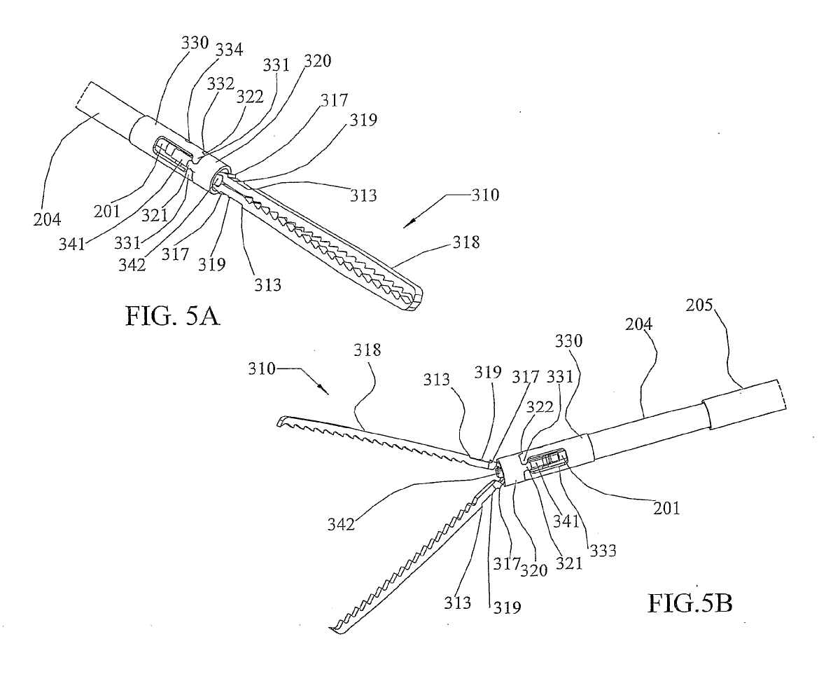

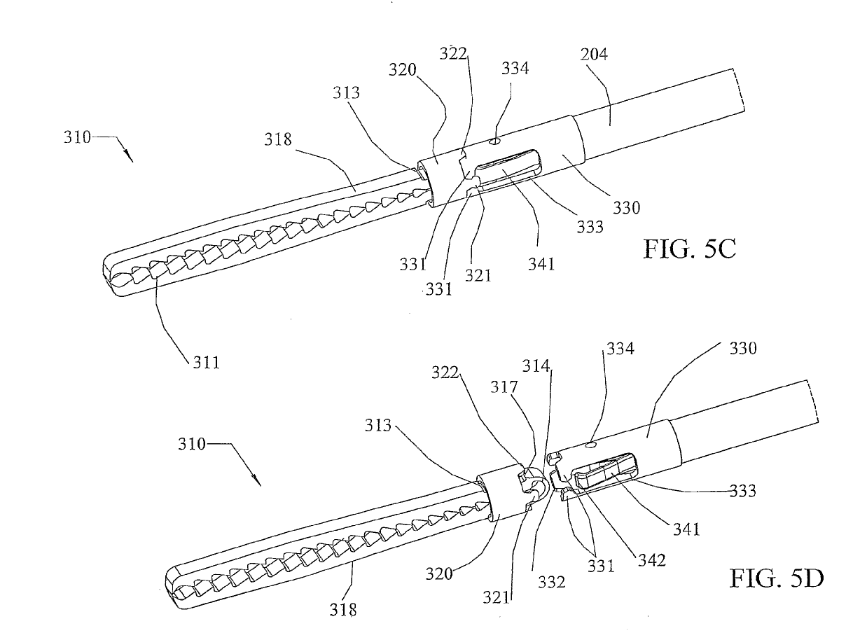

In FIG. 3B, fork 340 is seen in its most distal position. The fork cannot move further in the distal direction as it is being stopped by housing pin 334 of housing 330. Housing 330 is best seen in FIGS. 5A-5D.

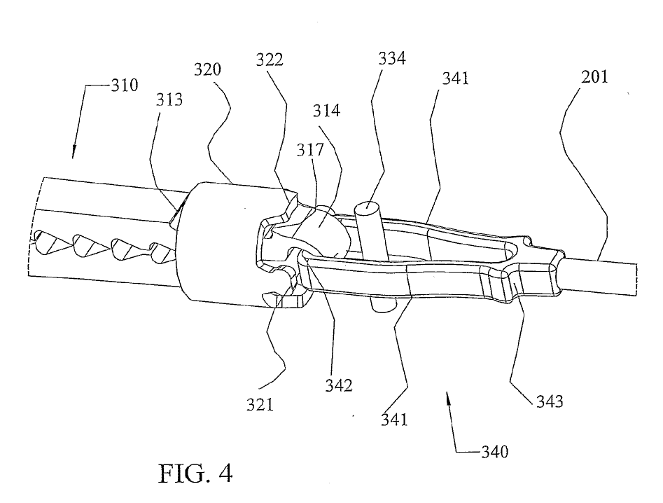

FIG. 4, to which reference is now made, illustrates how fork element 340 is attached to clip 310. Fork arm projections 342 are positioned within the loop-like region formed by clip hinge 314. The clip is shown in the Figure in its closed, but unlocked, position. In this Figure, fork arms 341 are positioned distally from release openings or slots 333; these openings or slots are not readily seen in the Figure but better seen elsewhere, for example, FIGS. 5B, 5C and 5D discussed below. Fork element 340 is in mechanical communication with control wire 201 within shaft 204 (FIGS. 1B and 1C). Activation of the system is effected as discussed in conjunction with FIGS. 1A-1D via wire 201. In FIG. 4, housing 330 of the applier's deployment assembly in which fork element 340 is positioned is not shown. Housing 330 applies a compressive force on fork arms 341 of force transmitting element 340, here a fork element. This force holds fork arm projections, also denoted herein occasionally as insertion elements, 342 of arms 341 within hinge 314.

FIGS. 5A-5D, to which reference is now being made, is here presented to provide for a better understanding of FIG. 4 and the method of the present invention. FIG. 5A is a view of the clip while it is still positioned in overtube 205 (not shown) during its insertion into a body lumen. The arms 341 of fork element 340 are visible and the fork arm projections 342 are positioned within the loop-like region formed by clip hinge 314. The male yoke members 321 of lock element 320 are mateably held by yoke elements 331 of housing 330 while the orientation teeth 322 of clip lock 320 is mateably held by recesses, herein denoted as housing orientation spaces 332 of housing 330.

Housing pin 334, best seen in FIGS. 4, 5C and 5D, which functions as a distal stop for fork element 340 also serves as an anti-rotation element for fork element 340 assuring that fork arms 341 are properly aligned with housing 330. It orients fork element 340 within housing 330 to ensure that fork arms 341 are positioned so that they are in a correct orientation relative to release openings (slots) 333 of housing 330 thus allowing clip disengagement as will be further discussed below. Pin 334 further serves as a stopper preventing fork element 340 from fully exiting clip lock element 320 in the distal direction which would lead to unintentional and premature disengagement of the clip.

FIGS. 5B and 5C, to which reference is now made, show two views of clip 310 after it has been exposed by pulling overtube 205 (FIG. 5B) in the proximal direction. The clip is shown in its biased open position in FIG. 5B. At this point, the endoscopist can repeatedly close and open the clip to position and reposition it around a lesion. In FIGS. 5B and 5C, fork arm projections 342 are positioned in the loop-like region formed by hinge 314 best seen in FIG. 4. Clip lock element 320 is still in mateable connection with housing 330. As noted above, one or more orientation teeth 322 orient the housing so that it is in proper mating position to mate with clip lock element 320. In FIG. 4, pin 334 is positioned at the distal end of fork element 340 allowing further movement of fork element 340 in the distal direction. Proximal stop projections 317 and distal stop projections 313 are clearly shown in FIGS. 4 and 5B. In FIG. 5B, fork arms 341 are distally positioned vis-à-vis release openings 333 and they are compressed by housing 330.

FIGS. 5C and 5D, to which reference is now made, show fork arms 341 first being brought adjacent to release openings 333 of housing 330 (FIG. 5C) and then being released from their compressed state, extending through release openings 333 (FIG. 5D). In FIG. 5C, fork arms 341 are just beginning to exit recess openings 333. After extending through release openings 333, fork arms 341 may be used to push against male yoke members 321 of clip lock element 320. This disengages clip lock element 320 from housing 330 by freeing male yoke members 321 from housing yoke elements 331. In FIG. 5D, clip 310, which is closed and locked, is entirely disengaged from housing 330. Clip lock element 320 has advanced passed middle stop projections 319 (obscured) resting between distal stop projections 313 and proximal stop projections 317 (FIGS. 5C and 5D and FIGS. 2C and 2D) in lock region, also denoted herein as lock socket, 316 (FIG. 2E).

It should be noted that FIG. 5A represents the first step in the use of the clip and clip lock element of the present invention and FIGS. 5C and 5D the penultimate and ultimate steps of the method. FIG. 5B represents only the first part of the intermediate stage of the method. Not shown here are the steps of repeatedly opening and closing the clip in attempts to satisfactorily position and clamp the tissue to be compressed.

In what herein is called, for reference, step A, fork element 340 of FIG. 4 is pulled via control wire 201 in the proximal direction. Fork element 340 moves in that direction but its arms 341 are never brought to a position completely adjacent to release openings 333 as in FIGS. 5C and 5D. This movement of fork element 340 in the proximal direction causes clip 310 to move further into clip lock element 320 forcing clip arms 318 to move from their spaced apart open position to their closed position as in FIG. 5C. If the positioning of clip 310 is unsatisfactory, it is not locked and the fork is not pulled further in the proximal direction. Rather, the endoscopist pushes wire 201, forcing fork 340 in the distal direction, for reference, denoted herein as step B. This causes clip 310 also to move in the distal direction and clip arms 318 to reopen and return to a position akin to FIG. 5B.

It should be remembered that in all the pushing and pulling of wire 201, clip lock element 320 is not moved. Clip 310 moves relative to lock element 320 as lock element 320 remains engaged to housing 330.

Repositioning of clip 310 on the tissue to be compressed is effected and the clip is again provisionally brought to its closed position described in step A. If the repositioning is satisfactory, control wire 201 pulls fork element 340 further in the proximal direction to its position in FIG. 5C where fork arms 341 are adjacent to release openings 333. There, they exit openings 333 and they may be used, if needed, to push male yoke members 321 of clip lock element 320 away from housing yoke elements 331. This causes clip lock element 320 and housing 330 to separate as in FIG. 5D. At separation, clip lock element 320 rests in lock socket, that is lock region, 316 between projections 313 and 317.

When repositioning and closing the arms as in step A, the endoscopist readily avoids inadvertently locking clip 310 with clip lock element 320. Before locking, the clip must be brought in the proximal direction so that lock element 320 is brought over middle stop projections 319, best seen in FIG. 2E, of clip 310. The endoscopist will notice the increase in force required to pull clip 310 when projections 319 are about to pass under lock element 320. This signals the endoscopist that continuing to pull on control wire 201 in the proximal direction will cause clip 310 to lock and disengage. Projections 319 represent the irreversible point in the locking of clip 310.

The embodiment discussed above indicates that fork arms 341, when emerging from release openings 333, may be used to push against and disengage clip lock element 320 from housing 330. However, it is contemplated that other methods may also be used to effect disengagement of lock element 320 from housing 330. It is even contemplated that lock element 320 may by itself disengage from housing 330 after full deployment. This may occur because proximal stop projections 317 of clip 310 slightly separate the two sides of housing yoke elements 331 once they pass lock element 320. This creates a large enough gap for male yoke members 321 of lock element 320 to disengage from housing yoke elements 331. In effect, the proximal end of the clip applies a force on the yoke connection encouraging disengagement of the locked clip.

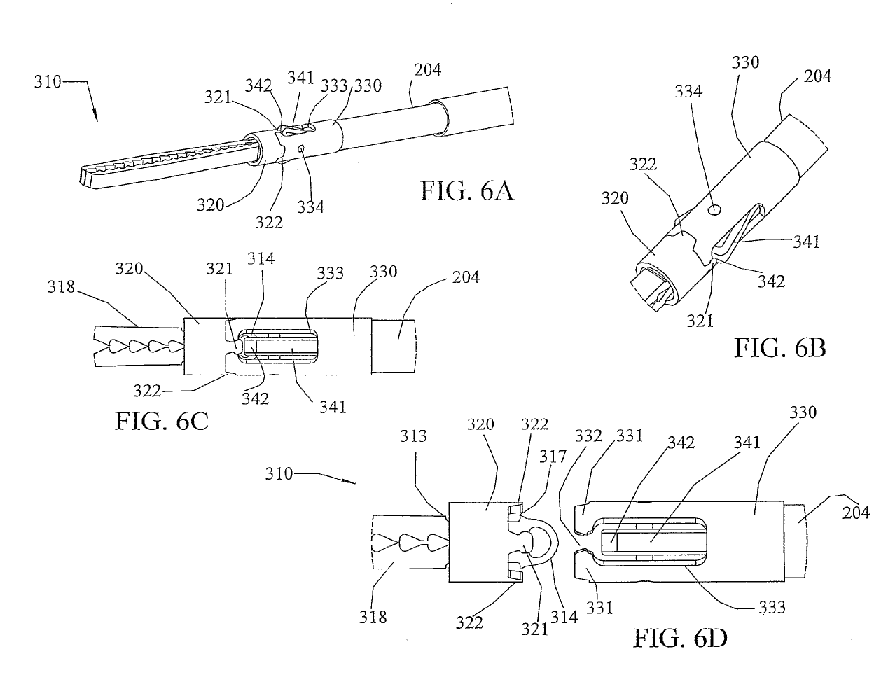

FIGS. 6A-6D, to which reference is now being made, show more detailed views than those shown in FIGS. 5C-5D. FIGS. 6A-6D are isometric and side views of the step of disengagement shown in FIGS. 5C and 5D. The numbered elements have all been discussed previously and accordingly will not be discussed again. FIGS. 6A-6D show fork arm projections 342 disengaged from clip hinge 314 and fork arms 341 moving through release openings, also denoted herein as release slots 333. FIG. 6D shows a side view of the totally disengaged closed and locked clip 310 with fork arms 341 extending through release openings 333. In FIG. 6D it can be seen that when the clip is locked clip lock element 320 is positioned forward of proximal stop projections 317 and up against distal stop projections 313. Middle stop projections 319 are obscured by clip lock 320 in the Figures.

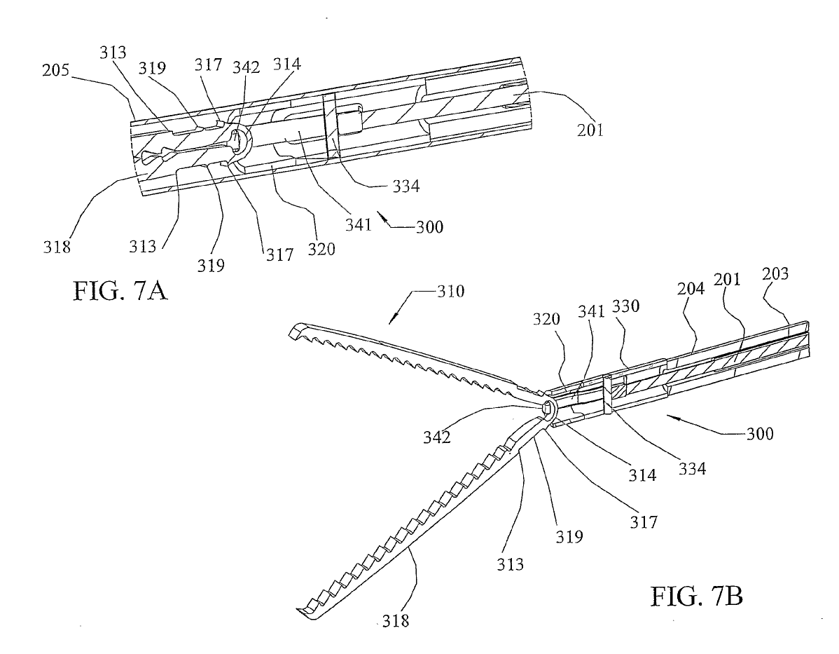

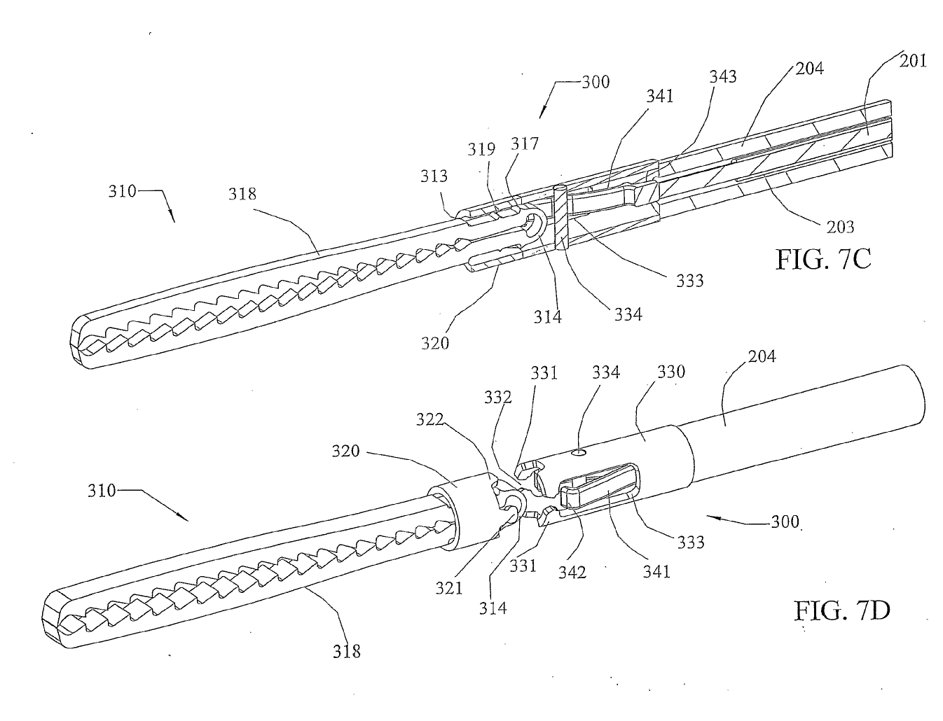

Reference is now made to FIGS. 7A-7D. FIGS. 7A-7C show various cut-away essentially side views of clip 310 and the applier's deployment assembly 300. FIG. 7D shows a side isometric view of the disengaged closed and locked clip 310 and deployment assembly 300. All of the elements, their construction and their operation, have been discussed previously and will not be discussed again. In FIGS. 7A and 7B, clip 310 is still engaged to clip deployment assembly 300 of the applier via fork arm projections 342. In FIG. 7C, the fork arm projections 342 have disengaged from clip hinge 314 and fork arms 341 are already exiting through release opening 333. In FIG. 7D, locked clip 310 is completely disengaged from deployment assembly 300 and fork arms 341 are extending out of release openings 333 in housing 330.

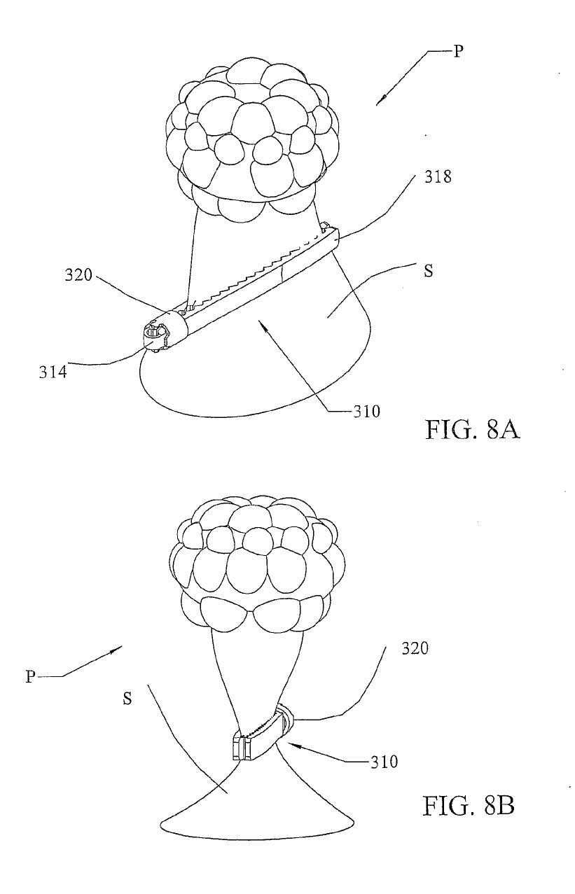

FIGS. 8A and 8B show two views of a locked compression clip assembly constructed according to an embodiment of the present invention and positioned on a stalk S of polyp P. Positioning, closing and locking of the clip may be effected as discussed previously. The construction of the clip may be as described above with reference to FIGS. 2A-7D. Opening and closing of the compression clip can be effected as often as needed to arrive at adequate positioning around the polyp. Only then would a user lock the clip assembly.

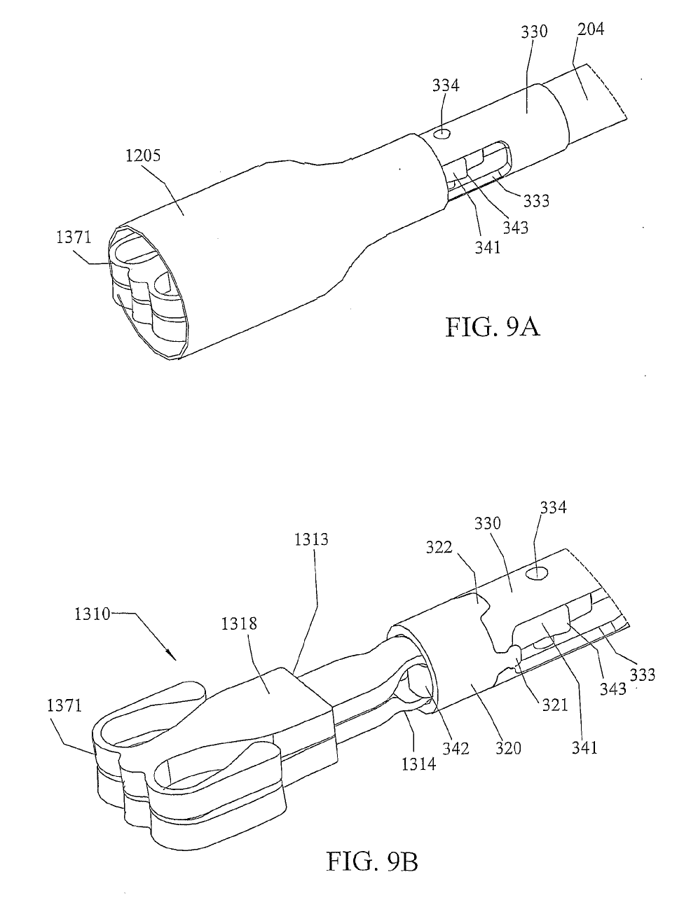

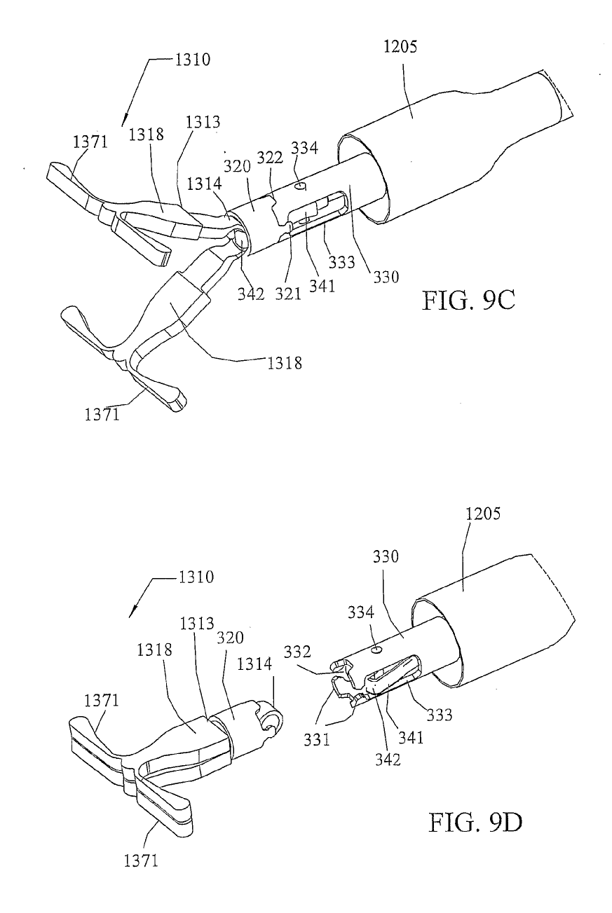

FIGS. 9A-9D show another embodiment of a clip constructed according to the present invention. Elements constructed and operative as in the clip embodiment of FIGS. 2A-7D have been given the same numbers. Equivalent, but slightly differently constructed, elements have been given a prefix digit of “1” with the number of the analogous part of the previous embodiment. Their function is essentially identical to the analogous part in the previous embodiment.

FIGS. 9A-9D show a clip 1310 with broad clip arms, that is clip elongate members, 1318, each arm having a clip arm projection 1371 at its distal end. Clip 1310 is constructed so as to have a broad surface area allowing better grasping of the tissue to be compressed. Overtube 1205 is of a non-uniform diameter with a broader distal end allowing the wider clip to be held in its closed position as it is advanced toward the tissue to be compressed. Overtube 1205 shown in FIG. 9A is truncated. It should readily be evident that it extends further in the proximal direction.

FIG. 9A shows clip 1310 in its unlocked closed position within overtube 1205 as it is advanced to the lesion. FIG. 9B shows clip 1310 in its unlocked closed position, as in FIG. 9A, but overtube 1205 is not shown. FIG. 9B shows clip 1310 engaged to deployment assembly housing 330 via fork arm projections 342 just as in the previously described embodiment. The substantially T-arm shaped distal end 1371 of clip 1310 is a gripping area. It has a larger gripping surface than in the previously described clip 310. FIG. 9C shows clip 1310 being exposed after pulling overtube 1205 in the proximal direction and opened so that clip arms 1318 are spaced apart in the clip's biased open position. FIG. 9D shows clip 1310 after being disengaged from fork arms 341 and housing 330. Disengagement is effected as in the embodiment discussed above. The disengaged clip in FIG. 9D is in its locked closed position.

In some versions of clip 1310, clip 1310 may have distal stop projections 1313, proximal stop projections 1317 and middle stop projections 1319 which function as their analogous parts in clip 310. In other versions of clip 1310, some of these projections may be absent as they may not be required. In these latter versions, geometry alone may prevent clip lock element 320 from sliding off clip 1310.

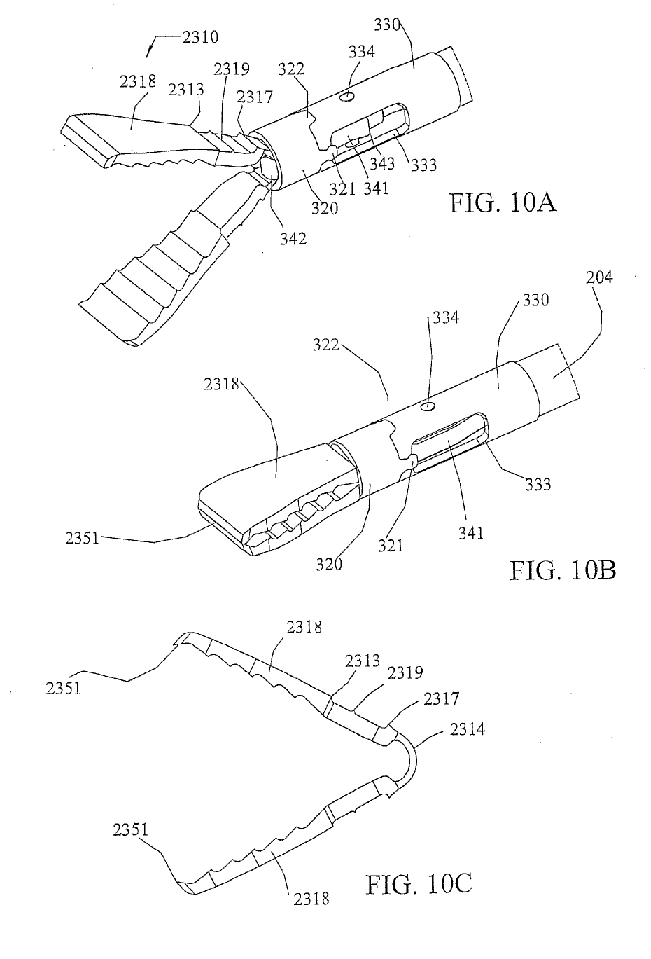

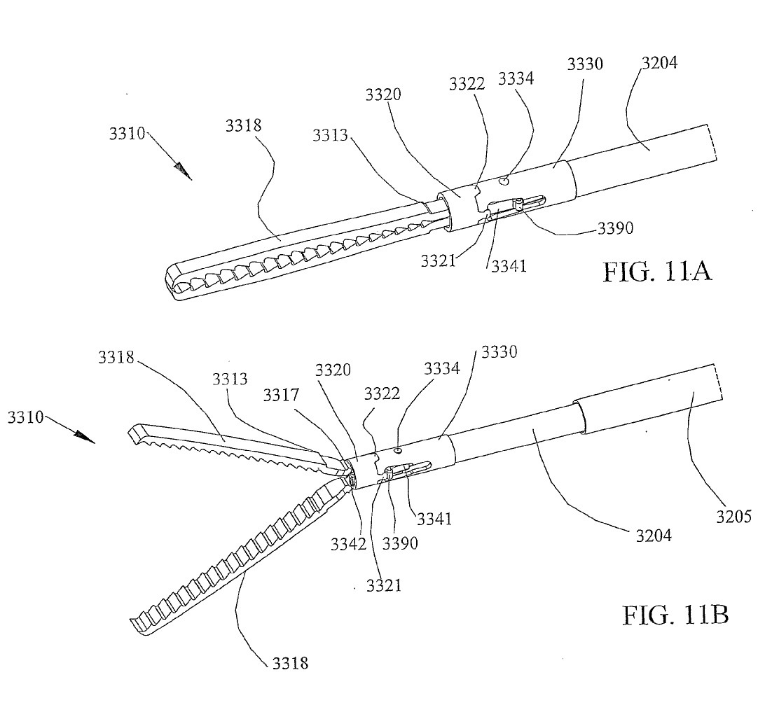

FIGS. 10A-10C show yet another clip constructed according to another embodiment of the present invention. Elements constructed and operative as in the clip embodiment of FIGS. 2A-7D have been given the same numbers. Equivalent, but slightly differently constructed, elements have been given a prefix digit of “2” with the number of the analogous part of the previous embodiment. Their function is essentially identical to the analogous parts in previous embodiments.

Clip 2310 is constructed as with the clip discussed in conjunction with FIGS. 2A-7D. The difference is essentially a slightly enlarged face at the distal end of clip 2310. Additionally, there is a distal protruding tooth 2351 at the distal end of each arm 2318 of the clip. Otherwise, the clip is constructed and operative as before including the presence of distal stop projections 2313, middle stop projections 2319 and proximal stop projections 2317. These projections function in locking and positioning the clip as described above in conjunction with the embodiment shown in FIGS. 2A-7D. In this embodiment, as in the previous embodiment, the overtube has a non-uniform diameter with a broader distal end; this overtube is not shown in the Figures.

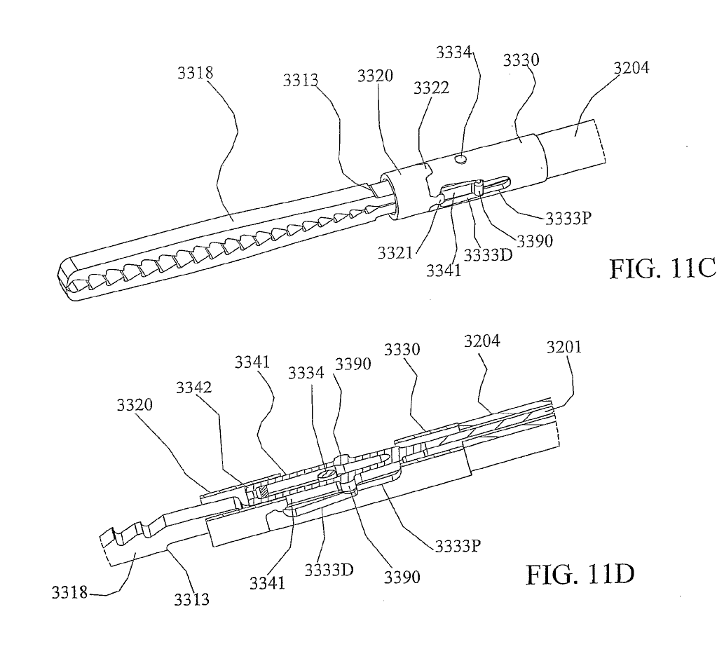

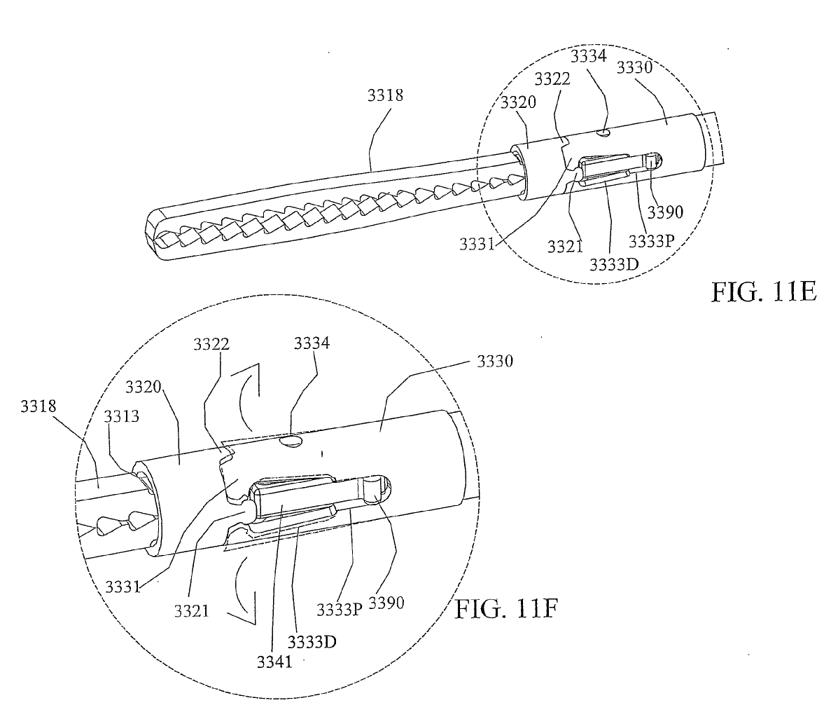

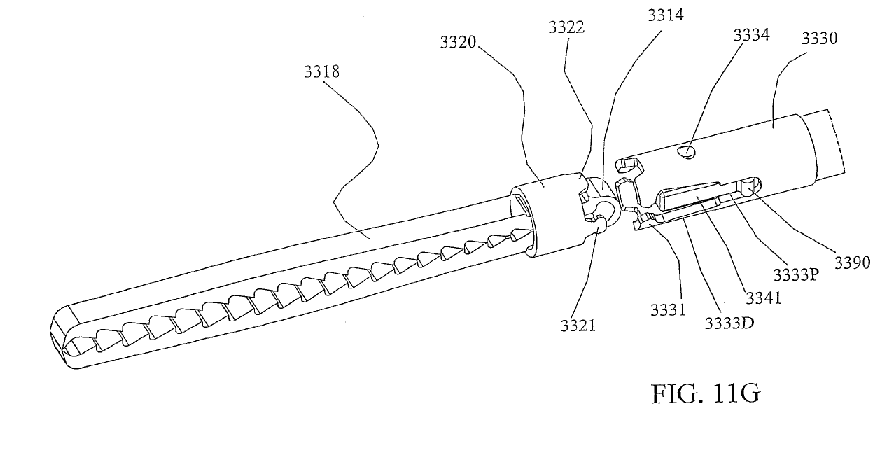

FIGS. 11A-11G, to which reference is now made, show yet another clip constructed according to an embodiment of the present invention applied in a slightly different manner. Elements constructed and operative as in the clip embodiment of FIGS. 2A-7D have been given the same numbers but with the addition of a prefix digit of “3”. Their function is essentially identical to the analogous parts in previous embodiments.

Clip 3310 and clip lock element 3320 are constructed as the clip and clip lock described in conjunction with FIGS. 2A-7D with the exception that there are no projections equivalent to middle stop projections 319 of clip 310. Middle stop projections 319 indicated the irreversible point in the locking process. The function of middle stop projections 319 in the present embodiment is provided by fork arm protrusions 3390 on fork arms 3341. Additionally, release slots 3333 in housing 3330 of the system are divided into wider slot regions 3333D at the distal end of the slots and narrower slot regions 3333P at the proximal end of the slots. This narrowing of the release slots creates a “step” which when encountered by fork arm protrusions 3390 indicates to the endoscopist that further motion of clip 3310 in the proximal direction will lock the clip. This “step” functions as middle stop projection 319 in clip 310. In order to pass the “step” in the proximal direction and lock the clip the user must provide a noticeably increased force. The “step” is a second type of “means for employing a resistive force operative to indicate that applying force to overcome the resistive force will lock the clip”.

Applying an increased force allows fork arm protrusions 3390 to slide in the proximal direction into the narrower slot end 3333P. If the endoscopist continues to pull control means, that is control wire, 3201 in the proximal direction, once clip 3310 is locked against distal stop projections 3313 situated on clip 3310, as in previous embodiments, fork element, that is force transmitting element, 3340 detaches from clip 3310 as fork arms 3341 emerge from slot 3333D. If made from a superelastic material, such as nitinol, fork arms 3341 may spring open; if arms 3341 are made from other resilient materials, such as stainless steel, the application of a force will slightly bend the arms leveraging their flexibility, so that fork arm projections 3342 can “exit” the clip's hinge loop.

The movement of protrusions 3390 in the narrow proximal end 3333P of the slots leads to a spreading of housing yoke elements 3331, as indicted by the diverging arrows in FIG. 11F. This spreading of housing yoke elements 3331 facilitates detachment of locked clip 3310 from housing 3330. If this is not enough to effect detachment, then, as in the other embodiments, fork element arms 3341 may be used to push against male yoke members 3321 of lock element 3320 to facilitate disengagement. As noted above, in this embodiment, fork element 3340, including its arms, may be made of any resilient material, not necessarily superelastic materials, having sufficient material strength.

In the above discussion of the present invention, the invention has been described as being used in bowel polyp resections. It should be evident to one skilled in the art that other types of lesions, in other organs in other organ systems, can also be resected using the present invention with little or no modification. Such organs include, but are not limited to, the urinary bladder and other organs of the urinary tract, the uterus, the liver, the esophagus, the gall bladder, the lungs and the rectum.

The ECC and system for employing the clip may also be used in closing perforations, naturally occurring or resulting from surgical procedures, and fistulas. For such types of lesions, the method of use of the system and clip is essentially the same as discussed above and shown in the Figures. The method may be modified slightly as the particular lesion warrants.

Additionally, the ECC and system discussed herein above may be used to effect hemostasis in all bleeding situations, not only those resulting from resected GI polyps or bleeding peptic ulcers. Resection of any organ that leads to bleeding or any blood vessels that have been ruptured or are otherwise leaking may be treated as described herein.

It should be readily apparent to one skilled in the art that the device and method of the present invention can be used to compress animal tissue as well as human tissue, particularly, but without limiting the invention, tissue of other mammalian species.

Although the invention has been described in conjunction with specific embodiments thereof, it is evident that many alternatives, modifications and variations will be apparent to those skilled in the art. Accordingly, the invention is intended to embrace all such alternatives, modifications and variations that fall within the spirit and broad scope of the appended claims. Also, it is to be understood that the phraseology and terminology employed herein is for the purpose of description and should not be regarded as limiting. In addition, citation or identification of any reference in this application shall not be construed as an admission that such reference is available as prior art to the present invention.

It will be appreciated by persons skilled in the art that the present invention is not limited by the drawings and description hereinabove presented. Rather, the invention is defined solely by the claims that follow.

1. A compression clip assembly for compressing tissue and operable by means of a user-operated applier, said assembly comprising:
A. an endoscopic compression clip having open and closed positions, wherein said clip includes:
(i) a pair of normally spaced apart elongate members each having an outward-facing surface, and having respective inward-facing opposing surfaces for holding and compressing tissue;
(ii) a hinge formed at least partly of a superelastic material and in operative mechanical connection with said elongate members; and
(iii) a lock region formed on said outer surface of each of said elongate members adjacent to said hinge, each said lock region being delimited by a first stop element proximate to said hinge and a second stop element distal from said hinge; and
B. a lock element for lockably engaging said lock regions so as to lock said clip in its closed position, said lock element and said lock regions being formed so as to facilitate relative translation therebetween until said lock element is positioned between said first and second stop elements.

2. A compression clip assembly according to claim 1, further including at least one third element in each lock region positioned between said first and second stop elements wherein locking of said clip is effected when said at least one third element is forcibly engaged by and passes within said lock element and wherein an increase in force is required for further movement of said clip through said lock element so as to lock said clip with said lock element.

3. A compression clip assembly according to claim 1, wherein said lock element further includes at least one orientation tooth and at least one male yoke member for disengageably mating with the user-operated applier.

4. A compression clip assembly according to claim 1, wherein said lock element locks said clip after being positioned against said second stop elements.

5. A compression clip assembly according to claim 1, wherein said hinge is configured as a substantially closed geometric shape enclosing an area large enough to accommodate a means for mechanical connection of the applier.

6. A compression clip assembly according to claim 1, wherein said elongate members of said clip are formed of a superelastic material.

7. A compression clip assembly according to claim 1, wherein when said clip is locked, the ratio of the length of said elongate members of said clip extending past said lock element to the length of said lock element is from about 1 to about 7.

8. A system for applying a compression clip for compressing tissue comprising:
A. a compression clip assembly, said assembly including:
(a) an endoscopic compression clip having open and closed positions wherein said clip includes:
(i) a pair of normally spaced apart, elongate members each having an outward-facing surface, and having respective inward facing opposing surfaces for holding and compressing tissue;
(ii) a hinge at least partly formed of a superelastic material and in operative mechanical connection with said elongate members; and
(iii) a lock region formed on said outer surface of each of said elongate members adjacent to said hinge, each lock region being delimited by a first stop element proximate to said hinge and a second stop element distal from said hinge; and
(b) a lock element lockably engaging said lock regions so as to lock said clip in its closed position, said locking element and said lock regions being formed so as to facilitate relative translation therebetween until said lock element is positioned between said first and second stop elements;
B. an applier which includes:
(a) a housing having a periphery with a pair of slots symmetrically positioned therein and disengageably mateable with said lock element;
(b) a force transmitting element positioned within said housing and including two arms formed of a resilient material, each of said arms having a free end and insertion elements formed thereat for insertion into said hinge;
(c) a control means operative to selectably move said force transmitting element in a direction of said first stop elements and in a direction of said second stop elements of said clip; and
C. means for applying a resistive force operative to indicate that applying force to overcome the resistive force will lock said clip,
wherein said control means may be selectably moved by a user in a selected one of the proximal and distal directions causing said force transmitting element to move by a preselected distance, the distance defined by the encounter of a resistive force when said clip is pulled in the proximal direction, the resistive force provided by the means for applying a resistive force so as to oppose movement of the clip within said lock element, and
wherein when said force transmitting element is pulled so as to move said clip beyond the preselected distance overcoming the increased resistive force, said clip is positioned so that said lock element locks said clip in its closed position and said pair of elongate members of said clip are positioned adjacent to each other thereby to compress tissue held therebetween, and said insertion elements pull away and disengage from said hinge and said force transmitting element arms exit said slots.

9. A system according to claim 8, wherein said lock element further includes at least one male yoke member and at least one orientation tooth and said housing further includes at least one yoke element and at least one housing orientation space for disengageably mating with said at least one male yoke member and said at least one orientation tooth, respectively.

10. A system according to claim 9, wherein said resilient material is a superelastic material and wherein when said force transmitting element is pulled so as to move beyond the preselected distance said force transmitting element arms, confined in said housing, are operative to disengage from said hinge and to spring open and exit said housing slots after disengaging from said clip.

11. A system according to claim 9, wherein when said force transmitting element is pulled so as to move beyond the preselected distance said force transmitting element arms disengage from said clip and then are positioned to push against said at least one male yoke member of said lock element, thereby disengaging said locked clip assembly from said housing of said applier.

12. A system according to claim 8, wherein said clip includes at least one third element, said at least one third element positioned between said first and said second stop elements and when encountered serves as said means for applying a resistive force, thereby indicating to the user imminent locking of said clip consequent to further application of force to said control means.

13. A system according to claim 8, wherein said arms of said force transmitting element include a pair of force transmitting element projections and each of said housing slots has a narrow proximal part and a wider distal part and at their junction said parts form a step, and when said pair of force transmitting element projections encounter said step it serves as a means for applying a resistive force, thereby indicating to the user imminent locking of said clip consequent to further application of force to said control means.

14. A system according to claim 13, wherein said housing includes at least one yoke element said housing being constructed of a material that allows spreading of said at least one housing yoke element when said force transmitting element projections enter said narrower proximal part of said slots so that said locked clip is more easily disengaged.

15. A system according to claim 8, where said resilient material of said force transmitting element arms is a superelastic material.

16. A system according to claim 8, wherein in said open position said clip forms an angle of at least about 45 degrees.

17. A system according to claim 8, wherein said applier further includes an overtube for compressing said elongate members of said compression clip holding them in said closed position while said clip is brought to tissue to be compressed.

18. A system according to claim 8, wherein said resilient material of said force transmitting element arms is a superelastic material and wherein when said force transmitting element is pulled so as to move past the preselected distance said force transmitting element arms spring open and exit said slots.

19. A method for compressing tissue comprising the steps of:
bringing a compression clip assembly, including a compression clip and a lock element, using an applier to tissue to be compressed;
opening and closing the compression clip, as often as necessary, around the tissue to be compressed until a proper positioning of the clip has been achieved;
locking the clip so that its elongate members are held adjacent to each other compressing the tissue held therebetween; and
freeing the locked clip from the applier by pulling on a compressed resilient force transmitting element of the applier so that it is brought to, and at least partly passes out of, slots in the wall of a housing of the applier removing the compressive force acting on the resilient force transmitting element allowing for disengagement of the clip from the applier.

20. A method according to claim 19, further including a step of drawing an overtube over the compression clip prior to said step of bringing and a step of pulling back the overtube and uncovering the clip, allowing the clip to return to its biased open position after said step of bringing.

21. A method according to claim 19, wherein said step of locking further includes a step of bringing the compression clip through the lock element so that the lock element passes over at least one projection on the clip after which the clip locks, the act of passing over the at least one projection after which the clip locks requiring additional force by a user signaling to the user that passing the at least one projection will irreversibly lock the clip.

22. A method according to claim 19, wherein said step of freeing further includes a step of moving the resilient force transmitting element so as to press against elements on the lock element mateable with elements on the housing of the applier to further assist in disengagement of the locked clip from the applier.

23. A method according to claim 19, wherein said step of locking further includes a step of bringing projections located on the resilient force transmitting element over a juncture formed by a narrower portion and a wider portion of the housing slots, the juncture requiring additional force by a user signaling to the user that passing the juncture will irreversibly lock the clip.

24. A method according to claim 23, wherein said step of freeing includes a step of pulling the resilient force transmitting element so that the projections thereon enter the narrower part of the housing slots thereby locking the clip and facilitating disengagement of the locked clip from the applier by spreading apart elements of the housing mateably engaged with elements of the lock element.
